(19) United States
(12) Patent Application Publication
(10) Pub. No.: US 2024/0115631 A1
(43) Pub. Date: Apr. 11, 2024

(54) COMPOSITION COMPRISING GRAIN-DERIVED LACTIC ACID BACTERIA FOR RELIEVING HANGOVER AND BOWEL TROUBLE

(71) Applicant: WELL-BEING LS CO., LTD, Gangneung-si, Gangwon-do (KR)

(72) Inventors: Jong Soon PARK, Gangneung-si (KR); Dong Jin LEE, Chuncheon-si (KR); Deuk Sik LEE, Gangneung-si (KR)

(73) Assignee: WELL-BEING LS CO., LTD, Gangneung-si, Gangwon-do (KR)

(21) Appl. No.: 17/767,702

(22) PCT Filed: Oct. 11, 2019
(86) PCT No.: PCT/KR2019/013394

§ 371 Date: Jun. 8, 2022

(30) Foreign Application Priority Data
Oct. 8, 2019 (KR) 10-2019-0124842

(51) Int. Cl. A61K 35/747; A61K 35/744; A61K 35/745; A61P 25/32

(52) U.S. Cl. CPC A61K 35/747; A61K 35/744; A61K 35/745; A61P 25/32

(57) ABSTRACT

The present invention relates to a composition for relieving hangover, the composition comprising the grain-derived lactic acid bacterium strain Lactobacillus fermentum JS (accession number/KCCM-10499) which exhibits a potential of increasing enzymatic activity of alcohol dehydrogenase (ADH) and aldehyde dehydrogenase (ALDH), and to a composite lactic acid bacterium composition for relieving hangover, the composition comprising the seven lactic acid bacterium species Lactobacillus plantarum, Lctobacillus casei, Lactobacillus rhamnosus, Lactobacillus acidophilus, Lactococcus lactis, Bifidobacterium breve, and Bifidobacterium lactis, which, although low in the potential of increasing enzymatic activity of ADH and ALDH, exhibits synergy effects when used in combination with the grain-derived lactic acid bacterium strain Lactobacillus fermentum JS (accession number/KCCM-10499).

### TECHNICAL FIELD

The present invention relates to a composition for hangover relief, which includes a grain-derived lactic acid bacterium [Lactobacillus fermentum JS (patent deposit/KCCM-10499)] strain having ADH (alcohol dehydrogenase) and ALDH (aldehyde dehydrogenase) enzyme activity and/or an ability of increasing the enzyme activity. Further, the present invention relates to a composition for hangover relief, which includes at least one lactic acid bacteria selected from the group consisting of Lactobacillus plantarum, Lactobacillus casei, Lactobacillus rhamnosus, Lactobacillus acidophilus, Lactococcus lactis, Bifidobacterium breve and Bifidobacterium lactis, so as to exhibit synergistic effects when used along with the grain-derived lactic acid bacterium [Lactobacillus fermentum JS (patent deposited/KCCM-10499)] strain, although the above bacteria have relatively low ability of increasing activities of ADH and ALDH enzymes.

### BACKGROUND ART

Hangover refers to a phenomenon in which the human body has specific discomfort, headache, or mental and physical decline in working capacity for 1 to 2 days after awakening from the sleep when having ingested alcohol. About 20% of alcohol entering the body is absorbed in the stomach, while most of the rest is absorbed in the small intestine, of which 80-90% is metabolized in the liver. It is known that the hangover is mostly caused by toxicity due to acetaldehyde as an intermediate metabolite formed in the process of decomposing alcohol in the liver.

Among commonly used methods to relieve hangovers, there is a way to reduce a concentration of alcohol remaining in the human body by supplying water, or to discharge alcohol out of the body by drinking something with diuretic effects such as coffee, tea, etc.

Several products for hangover relief are on the market, and most of such hangover relievers adopt a way to indirectly prevent and relieve hangovers by preventing damage to liver cells that decompose alcohol.

ADH (alcohol dehydrogenase) and ALDH (aldehyde dehydrogenase) are enzymes directly involved in the process of metabolizing alcohol consumed by the human body. During alcohol intake, the alcohol absorbed in the stomach and small intestine is decomposed by the liver. At this time, ADH (alcohol dehydrogenase) oxidizes alcohol into highly toxic acetaldehyde, and the formed acetaldehyde is converted into acetic acid having very weak toxicity by ALDH (aldehyde dehydrogenase) through a metabolic process, thereby decomposing the alcohol.

In humans, ADH (alcohol dehydrogenase) and ALDH (aldehyde dehydrogenase) activities vary according to birth, age, and conditions when drinking and ingesting alcohol. Specifically, if the ALDH (aldehyde dehydrogenase) activity is poor, the toxicity of acetaldehyde causes feeling stronger and longer hangover.

Meanwhile, alcohol metabolism has previously been known that alcohol is absorbed in the stomach and small intestine and then decomposed in the liver, but recently, it is discovered that alcohol is also decomposed by beneficial microorganisms in the small intestine, which in turn leads to a growing interest.

Lactobacillus is the most well-known microorganism among beneficial bacteria, is commonly referred to as probiotics in Korea, and has effects of inhibiting the growth of harmful bacteria in the human intestine. In other words, lactic acid bacteria do not directly inhibit harmful bacteria but secrete lactic acid bacteria-producing substances called postbiotics, thereby creating an environment where harmful bacteria cannot live and eventually suppressing harmful bacteria.

Further, some of the lactic acid bacteria secrete ADH (alcohol dehydrogenase) and ALDH (aldehyde dehydrogenase) on their own to help metabolize alcohol in the small intestine and, even in the case of a strain that does not directly secrete ADH (alcohol dehydrogenase) and ALDH (aldehyde dehydrogenase), the strain may secrete a substance that can act as a coenzyme among lactic acid bacteria-producing substances called postbiotics or intracellular useful substances of the strain, and thus may increase ADH (alcohol dehydrogenase) and ALDH (aldehyde dehydrogenase) activities. Most of these lactic acid bacteria-producing substances and intracellular useful substances of lactic acid bacteria have very stable properties against heat and acid because of small molecular weight and very strong binding force.

Therefore, when ingested lactic acid bacteria having alcohol dehydrogenase (ADH) and aldehyde dehydrogenase (ALDH) secreting ability or lactic acid bacteria containing intracellular useful substances and lactic acid bacteria-producing substances that can increase ADH (alcohol dehydrogenase) and ALDH (aldehyde dehydrogenase) activities, it is known that these bacteria are helpful for decomposing alcohol in the liver and thus reduce an amount of alcohol delivered to the liver, thereby being helpful for relieving hangover. Although related products are being produced, there is a problem of insufficient effects.

On the other hand, many people suffer from intestinal problems such as abdominal pain, nausea and diarrhea along with hangover after drinking alcohol, however, there is still no product that solves such intestinal problems.

### DISCLOSURE

### Technical Problem

The main prior art related to hangover relief is focused on maintaining the activity of ADH (alcohol dehydrogenase) and ALDH (aldehyde dehydrogenase) secreted in the human body by preventing damage to hepatocytes that decompose alcohol.

However, this method has a disadvantage in that alcohol and acetaldehyde cannot be quickly decomposed when the activity of ADH (alcohol dehydrogenase) and ALDH (aldehyde dehydrogenase) secreted by an individual is low, or when an excessive amount of alcohol is consumed, hence making it difficult to actually sense effects of the above method.

Accordingly, an object of the present invention is to develop lactic acid bacteria having ADH (alcohol dehydrogenase) and ALDH (aldehyde dehydrogenase) secretion performance or lactic acid bacteria that contain a lactic acid bacteria-producing substance and an intracellular useful substance capable of increasing ADH (alcohol dehydrogenase) and ALDH (aldehyde dehydrogenase) activities, and to provide a composition for hangover relief with excellent effects, which may decompose alcohol in the small intestine and thus reduce an amount of alcohol delivered to the liver when drinking alcohol, thereby being helpful for relieving hangover.

Further, another object of the present invention is to provide a composition for hangover relief that can relieve intestinal problems (or bowel troubles) such as abdominal pain, nausea, diarrhea, etc. occurring along with the hangover.

### Technical Solution

In order to achieve the above objects,

the present invention provides a composition for hangover relief, including: a grain-derived lactic acid bacterium [Lactobacillus fermentum JS (patent deposited/KCCM-10499)] strain which has an ability of secreting alcohol dehydrogenase (“ADH”) and aldehyde dehydrogenase (“ALDH”) enzymes and/or increasing activities of these enzymes.

Further, the present invention provides a composition for hangover relief, including: a grain-derived lactic acid bacterium [Lactobacillus fermentum JS (patent deposited/KCCM-10499)] strain; and one or more species of lactic acid bacteria selected from the group consisting of Lactobacillus plantarum, Lactobacillus casei, Lactobacillus rhamnosus, Lactobacillus acidophilus, Lactococcus lactis, Bifidobacterium breve and Bifidobacterium lactis, which exhibit synergistic effects when mixed with Lactobacillus Fermentum JS (patent deposited/KCCM-10499) strain although the ability of increasing ADH and ALDH enzyme activities is weak or is not involved.

### Advantageous Effects

In the present invention, a composition for hangover relief that includes a grain-derived lactic acid bacterium [Lactobacillus fermentum JS (patent deposited/KCCM-10499)] strain, or a composite lactic acid bacteria composition for hangover relief that includes a grain-derived lactic acid bacterium [Lactobacillus fermentum JS (patent deposited/KCCM-10499)] strain and one or more species of lactic acid bacteria selected from the group consisting of Lactobacillus plantarum, Lactobacillus casei, Lactobacillus rhamnosus, Lactobacillus acidophilus, Lactococcus lactis, Bifidobacterium breve and Bifidobacterium lactis may secrete enzymes such as ADH and ALDH involved in alcohol metabolism that occurs in the small intestine and the liver, or may increase activities thereof, thereby directly acting on alcohol and acetaldehyde to decompose the same, so that the hangover can be relieved while not being uneasy on the small intestine or the liver.

Further, the composition for hangover relief of the present invention is effective in relieving intestinal problems such as abdominal pain, nausea, diarrhea, etc. occurring after drinking alcohol.

### DESCRIPTION OF DRAWINGS

FIG. 1 shows the whole genome contig of the selected strain [Lactobacillus fermentum JS (patent deposit/KCCM-10499)].

FIG. 2 illustrates 16S rDNA nucleotide sequence of Lactobacillus fermentum JS-KCCM-10499.

FIG. 3 shows measured results of ADH and ALDH activities of the selected strain [Lactobacillus fermentum JS (patent deposit/KCCM-10499)].

FIG. 4 shows measured results of ADH and ALDH activities of the mixed strain.

FIG. 5 shows measured results of alcohol reduction change over time of Soju (Korean alcoholic beverage) to which the product of the present invention was added.

### BEST MODE

According to most preferred embodiments of the present invention, there is proposed a composition for hangover relief, which includes a grain-derived lactic acid bacterium [Lactobacillus fermentum JS (patent deposited/KCCM-10499)] strain having an ability of secreting ADH and ALDH enzymes and/or increasing activities of these enzymes.

### Detailed Description of Preferred Embodiments of Invention

The inventors of the present invention have tried to find lactic acid bacteria that secrete ADH and ALDH as enzymes involved in alcohol metabolism occurring in the small intestine and the liver or increase activities thereof, as well as a composition for hangover relief that includes the above lactic acid bacteria, and have discovered that Lactobacillus fermentum JS (KCCM-10499) secretes ADH and ALDH or increases activities thereof, thereby completing the present invention.

In other words, the present invention provides a composition for hangover relief, which includes a grain-derived lactic acid bacterium [Lactobacillus fermentum JS (patent deposited/KCCM-10499)] strain having an ability of secreting ADH and ALDH enzymes and/or increasing activities of these enzymes.

The composition for hangover relief of the present invention may include Lactobacillus fermentum JS (KCCM-10499)] that secretes ADH and ALDH, which are enzymes involved in alcohol metabolism in the small intestine and the liver, or increases activities of the above enzymes. Therefore, unlike the existing hangover relievers in an aspect of mechanism, the composition of the present invention may act directly on alcohol and acetaldehyde to decompose the same so that an amount of alcohol delivered to the liver is reduced, thereby relieving the hangover while not being uneasy on the small intestine or the liker.

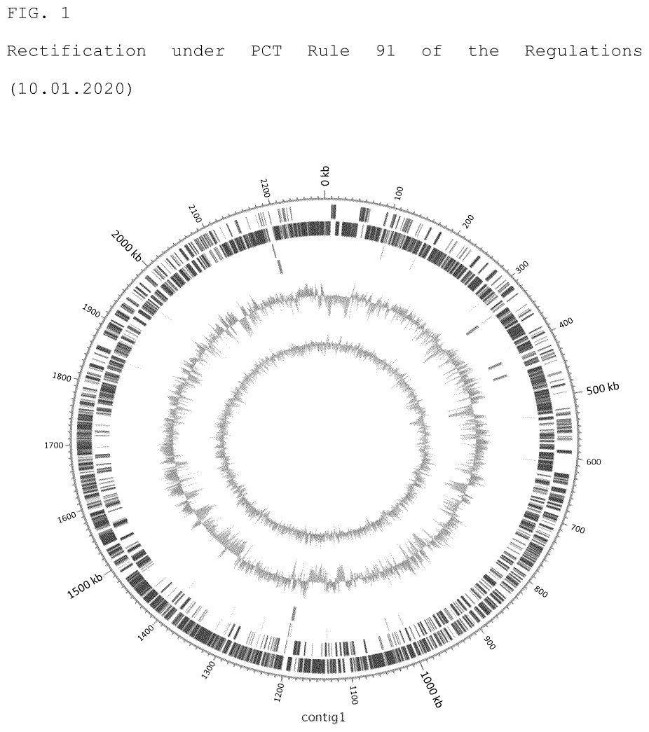

FIG. 1 shows the whole genome contig of Lactobacillus fermentum JS (Patent Deposit/KCCM-10499) of the present invention (See Registration Patent 10-0435168).

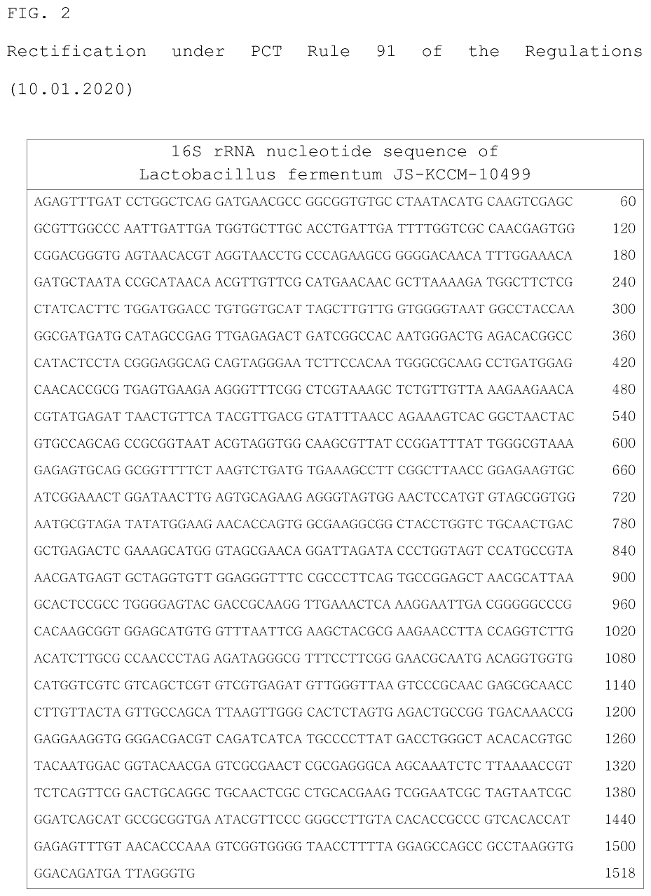

FIG. 2 illustrates 16S rDNA nucleotide sequence of Lactobacillus fermentum JS-KCCM-10499.

On the other hand, the present inventors found that some lactic acid bacteria have a little low ability to secrete ADH and ALDH enzymes or to increase activities thereof, but exhibit synergistic effects when used together with the grain-derived lactic acid bacterium [Lactobacillus fermentum JS (patent deposited/KCCM-10499)] strain, therefore, have completed the present invention.

That is, the present invention provides a composite lactic acid bacteria composition for hangover relief that includes a grain-derived lactic acid bacterium [Lactobacillus fermentum JS (patent deposited/KCCM-10499)] strain and one or more species of lactic acid bacteria selected from the group consisting of Lactobacillus plantarum, Lactobacillus casei, Lactobacillus rhamnosus, Lactobacillus acidophilus, Lactococcus lactis, Bifidobacterium breve and Bifidobacterium lactis.

The composition for hangover relief of the present invention as described above, the bacteria are living or dead bacteria, or may contain a product thereof.

The one or more species of the bacteria selected from the group consisting of Lactobacillus plantarum, Lactobacillus casei, Lactobacillus rhamnosus, Lactobacillus acidophilus, Lactococcus lactis, Bifidobacterium breve and Bifidobacterium lactis is not related to the strain.

Unlike existing hangover relieving agents that provide only effects of protecting the liver, the composition for hangover relief of the present invention may relieve the hangover caused by drinking alcohol and improve intestinal problems (or bowel troubles) such as diarrhea, abdominal pain, intestinal discomfort, etc. through Lactobacillus fermentum JS (KCCM-10499), as a strain that secretes ADH and ALDH or increases activities thereof (or activates ADH and ALDH) which are enzymes involved in alcohol metabolism occurring in the small intestine and the liver. That is, the composition for hangover relief of the present invention is mechanically different from the existing hangover relieving agents and may act directly on alcohol and acetaldehyde to decompose the same, so as to effectively relieve the hangover while not being uneasy on the small intestine or the liver.

Alternatively, the composition for hangover relief of the present invention may include, in addition to the Lactobacillus fermentum JS (KCCM-10499) as described above, one or more species of lactic acid bacteria selected from the group consisting of Lactobacillus plantarum, Lactobacillus casei, Lactobacillus rhamnosus, Lactobacillus acidophilus, Lactococcus lactis, Bifidobacterium breve and Bifidobacterium lactis that have a little low ability to increase activities of ADH and ALDH enzymes but exhibit synergistic effects when used together with the grain-derived lactic acid bacterium [Lactobacillus fermentum JS (patent Deposited/KCCM-10499)], thereby achieving hangover relief and prevention of bowel troubles as well as inherent effects of probiotics.

It is believed that the above synergistic effects may be obtained because the mixed strains appropriately stimulate the lactic acid bacterial strains having physiologically active effects, so as to produce physiologically active substances much more through competition between the bacteria.

The strains use in the present invention are purchased among commercial strains under production and sale, or may be grown by any typical culture method for lactic acid bacteria, collected or recovered by an isolation process such as centrifugation and, but not limited to, produced and used in the form of probiotics by lyophilization. A process for production of lactic acid bacteria powder through lyophilization may include mixing a strain concentrate and starch, lyophilizing the mixture to produce the powder and, optionally, adding a supplementary material such as glucose, fructose, etc.

The composition for hangover relief of the present invention preferably includes the Lactobacillus fermentum JS (patent deposit/KCCM-10499) strain, as well as two (2) or more species of lactic acid bacteria selected from the group consisting of Lactobacillus plantarum, Lactobacillus casei, Lactobacillus rhamnosus, Lactobacillus acidophilus, Lactococcus lactis, Bifidobacterium breve and Bifidobacterium lactis, and more preferably, includes the Lactobacillus fermentum JS (patent deposit/KCCM-10499) strain, as well as seven (7) species of lactic acid bacteria including Lactobacillus plantarum, Lactobacillus casei, Lactobacillus rhamnosus, Lactobacillus acidophilus, Lactobacillus lactis, Bifidobacterium breve and Bifidobacterium lactis.

The Lactobacillus fermentum JS strain and the lactic acid bacteria exhibiting synergistic effects when used together with the Lactobacillus fermentum JS strain, which are included in the composition for hangover relief, may have excellent ethanol tolerance and excellent ethanol and/or acetaldehyde decomposition ability, in addition, superior acid tolerance, bile acid resistance and intestinal fixation.

The hangover relief composition described above may be for use in removing one or more of ethanol and acetaldehyde from a sample. The term “removal” refers to decreasing a concentration of one or more of ethanol and acetaldehyde in a sample, and may include completely removing the above materials. The sample may be body fluid. The sample may be intestinal fluid or blood. The intestinal fluid may be gastric juice, duodenal fluid, small intestine fluid, or large intestine fluid.

In the composition for hangover relief including, in addition to the Lactobacillus fermentum JS (KCCM-10499), one or more species of lactic acid bacteria selected from the group consisting of Lactobacillus plantarum, Lactobacillus casei, Lactobacillus rhamnosus, Lactobacillus acidophilus, Lactococcus lactis, Bifidobacterium breve and Bifidobacterium lactis, 40 to 60% by weight (“wt. %”) of the Lactobacillus Fermentum JS (KCCM-10499) and 40 to 60 wt. % of one or more species of lactic acid bacteria selected from the group consisting of Lactobacillus plantarum, Lactobacillus casei, Lactobacillus rhamnosus, Lactobacillus acidophilus, Lactococcus lactis, Bifidobacterium breve and Bifidobacterium lactis to a total weight of the hangover relief composition are preferably included.

Although the ability of increasing the activities of ADH and ALDH enzymes is insignificant or does not exist, the lactic acid bacteria exhibiting synergistic effects when used together with the grain-derived lactic acid bacteria Lactobacillus fermentum JS (patent deposit/KCCM-10499) strain may include: 8 to 12 wt. % of Lactobacillus plantarum; 8 to 12 wt. % of Lactobacillus casei; 6 to 10 wt. % of Lactobacillus rhamnosus; 4 to 8 wt. % of Lactobacillus acidophilus; 4 to 8 wt. % of Lactococcus lactis; 3 to 7 wt. % of Bifidobacterium breve; and 3 to 7 wt. % of Bifidobacterium lactis, to a total weight of the hangover relief composition.

The present invention provides a product for hangover relief, which includes a hangover relief composition.

The hangover relief product of the present invention may be separately ingested before drinking an alcoholic beverage, but is preferably ingested with an alcoholic beverage by adding the same to the alcoholic beverage during drinking.

In this case, compared to other products that are ingested separately from an alcoholic beverage before or after drinking, the inventive product may directly decompose alcohol contained in the alcoholic beverage without changing the taste, color and turbidity thereof, and may further decompose acetaldehyde along with alcohol when ingested together with the alcoholic beverage, thereby attaining excellent effects of quickly relieving the hangover.

The hangover relief product according to the present invention may be prepared in any form of different foods, for example, beverages, teas, vitamin complexes, powders, granules, tablets, capsules, and the like.

In one embodiment, the hangover relief product is not particularly limited except for containing a hangover relief composition, and may include various sweeteners or natural carbohydrates as an additional component such as conventional food. That is, as natural carbohydrates, monosaccharides such as glucose and fructose, disaccharides such as sucrose, maltose, lactose, etc., conventional sugars such as starch, dextrin, cyclodextrin, etc., fructo-oligosaccharides, palatinose,

indigestible maltodextrin, and sugar alcohols such as xylitol, sorbitol, erythritol, etc. may be included. Examples of the sweeteners may include natural sweeteners (thaumatin, stevia extract, glycyrrhizin, etc.) and synthetic sweeteners (saccharin, aspartame, etc.).

In addition, the product of the present invention may include various nutrients, vitamins, minerals (electrolytes), flavoring agents such as synthetic flavoring agents and natural flavoring agents, coloring agents, pectic acid and salts thereof, alginic acid and salts thereof, organic acids, protective colloidal thickeners, pH adjusters, stabilizers, preservatives, glycerin, alcohol, carbonation agents used in carbonated beverages, and the like.

The present invention preferably includes at least one selected from starch, glucose, fructose, fructo-oligosaccharide, isomalto-oligosaccharide, indigestible maltodextrin and palatinose.

When the hangover relief product of the present invention is formed of powder, the powdered hangover relief composition of the present invention and other additional ingredients may be pulverized and mixed.

The hangover relief product may include 40 to 60 wt. % of the strain, 5 to 30 wt. % of glucose or fructose, 5 to 20 wt. % of fructo-oligosaccharide or isomalto-oligosaccharide, 5 to 20 wt. % of indigestible maltodextrin and 1 to 3 wt. % of palatinose mixed together, whereby the number of final viable cells may range from 1.0×10⁸ to 9.0×10⁹ cfu/g.

Hereinafter, the present invention will be described in more detail by ways of examples. However, these examples are for illustrative purposes only and the scope of the present invention is not limited thereto.

### <Example 1> Confirmation of Alcohol-Tolerant Strain

In order to be effective in relieving hangovers, an alcohol-tolerant strain was selected because the strain could be proliferated in the small intestine with an increased alcohol concentration. A medium used for alcohol tolerance of the strain was prepared by adding 5.5% of MRS (de Man, Rogosa & Sharpe) medium powder to fermented spirits diluted with alcohol concentrations of 10%, 15%, 20%, 25%, and 30% to dissolve the same, followed by sterilizing 20 ml of this liquid medium through a membrane filter.

After inoculating 1% of freeze-dried candidate strains at a concentration of 1.0×10⁹ cfu/g in MRS medium mixed with fermented alcohol at each concentration, the product was subjected to standing culture at 37° C. so as to determine alcohol tolerance based on a degree of growth in the MRS medium mixed with fermented alcohol compared to normal MRS medium. The growth degree of the strain was determined by measuring an absorbance of 1 ml of the sample at 610 nm using a UV-VIS spectrophotometer, wherein each sample was collected in an amount of 1 ml after 2 hours.

All strains used for selection are GRAS (Generally Recognized As Safe) grade lactic acid bacteria, and only the lactic acid bacteria able to be used for food were selected and used from the Food and Drug Administration's Food Ingredients List. Further, among 20 kinds of grain-derived lactic acid bacteria owned/deposited by Wellbeing LS Co., Ltd. and 50 kinds of public-announced general strains, 8 species of strains with relatively higher alcohol tolerance were selected and shown in Table 1. Table 1 shows test results of the top 4 strains having alcohol tolerance of public-announced/vegetable-origin lactic acid bacteria among candidate strains.

TABLE 1

| | | Alcohol concentration of medium | | | | |
|---|---|---|---|---|---|---|
| Name of candidate strain | | (v/v %) | | | | |
| (deposit no.) | | 10 | 15 | 20 | 25 | 30 |
| Public- | Lactobacillus plantarum | +++ | ++ | + | − | − |
| announced | (KCCM 11322) | | | | | |
| Public- | Lactobacillus fermentum | ++ | ++ | + | − | − |
| announced | (KCCM 35461) | | | | | |
| Public- | Weissella cibaria | ++ | ++ | + | + | − |
| announced | (KCCM 41287) | | | | | |
| Public- | Lactobacillus acidophilus | +++ | ++ | + | − | − |
| announced | (KCCM 32820) | | | | | |
| Vegetable- | Weissella cibaria LS-33 | +++ | ++ | + | − | − |
| origin | (KCCM 80198) | | | | | |
| Vegetable- | Lactobacillus fermentum | +++ | +++ | +++ | +++ | +++ |
| origin | JS (KCCM 10499) | | | | | |
| Vegetable- | Lactobacillus plantarum | ++ | ++ | + | − | − |
| origin | LS-65 (KCCM 80199) | | | | | |
| Vegetable- | Lactobacillus fermentum | ++ | ++ | + | − | |
| origin | LS-501 (KCCM 80201) | | | | | |

*Growth compared to normal MRS medium (control)
−: 50% or less,
+: 50% or more,
++: 75% or more,
+++: 100

### <Example 2> Measurement of ADH (Alcohol Dehydrogenase) and ALDH (Aldehyde Dehydrogenase) Activities of the Top 8 Strains Having Alcohol Tolerance

ADH activity was determined by measuring an absorbance at 340 nm of NADH (Nicotinamide adenine dinucleotide, reduced form) formed during decomposition of alcohol.

To a 15 ml Cornical tube, 100 μl of 99% alcohol, 500 μl of aqueous NAD (Nicotinamide adenine dinucleotide) solution, and 100 μl of a candidate strain at a concentration of 1.0×10⁸ cfu/ml were added, followed by further adding 1.1 ml of 0.01M glycine-NaOH buffer (pH 8.8) to make a total volume of 1.8 ml and then reacting the same in a constant-temperature water bath at 25° C. for 10 minutes. After the reaction was completed, 250 μl of ADH was added to measure a change in absorbance at 340 nm, whereas distilled water was added instead of the sample in a control group. For a positive control group, Hepos syrup manufactured by Cho-A Pharmaceutical was purchased at a pharmacy and diluted by 1/2 according to the corresponding prescription. The ADH activity was expressed as a ratio of the maximum absorbance at the end of the reaction to the maximum absorbance of the control group, and was calculated by the following equation.

ADH activity=(B/A)×100
A: maximum absorbance of the control group
B: maximum absorbance of the experimental group

Determination of ALDH Activity

As for ALDH activity, the change in absorbance according to the production of NADH was measured at 340 nm. Specifically, 2.1 ml of distilled water, 100 μl of 1M tris HCl, 100 μl of 3M KCl, 100 μl of the candidate strain at a concentration of 1.0×10⁸ cfu/ml, 100 μl of NAD, 100 μl of mercaptoethanol and 100 μl acetaldehyde were mixed in a 15 ml Cornical tube, followed by reacting at 25° C. for 10 minutes and then adding 100 μl of ALDH thereto so as to measure a change in absorbance at 340 nm. At this time, for a control group, distilled water was added instead of the candidate strain. A positive control group used herein was a 1/2 diluted solution of the same Hepos syrup manufactured by Cho-A Pharmaceutical as used in the measurement of effects of ADH activity. In this regard, ALDH activity was expressed as a ratio of the maximum absorbance at the end of the reaction to the maximum absorbance of the control group, and was calculated by the following equation.

ALDH activity=(B/A)×100
A: maximum absorbance of the control group
B: maximum absorbance of the experimental group

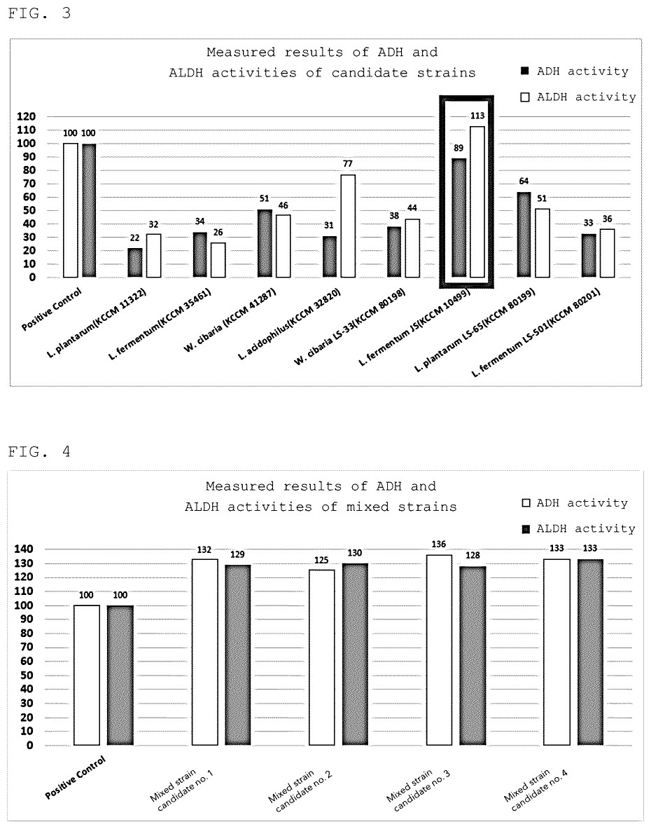

The results are shown in FIG. 3.

As a result of the experiment, among the top 8 strains having alcohol tolerance for which the measurement of ADH activity and ALDH activity was completed, Lactobacillus fermentum JS (patent deposit/KCCM-10499) strain having high alcohol tolerance and high ADH and ALDH activities was determined as the finally selected strain, and the present invention was completed.

Enzymatic properties (API 50 CHL KIT) of the selected strain Lactobacillus fermentum JS (patent deposit/KCCM-10499) are shown in Table 2 below.

TABLE 2

| | | |
|---|---|---|
| 0 | Control | − |
| 1 | Glycerol | − |
| 2 | Ertythritol | − |
| 3 | D-Arabinose | − |
| 4 | L-Arabinose | + |
| 5 | Ribose | + |
| 6 | D-Xylose | − |
| 7 | L-Xylose | − |
| 8 | Adonitol | − |
| 9 | β Methyl-xyloside | − |
| 10 | Galactose | + |
| 11 | D-Glucose | + |
| 12 | D-Fructose | + |
| 13 | D-Mannose | − |
| 14 | L-sorbose | − |
| 15 | Rhamnose | − |
| 16 | Dulcitol | − |
| 17 | Inositol | − |
| 18 | Mannitol | − |
| 19 | Sorbitol | − |
| 20 | α Methyl-D-mannoside | − |
| 21 | α Methyl-D-glucoside | − |
| 22 | N Acetyl glucosamine | − |
| 23 | Amygdaline | − |
| 24 | Arbutine | − |
| 25 | Esculine | − |
| 26 | Salicine | − |
| 27 | Cellobiose | − |
| 28 | Maltose | + |
| 29 | Lactose | + |
| 30 | Melibiose | + |
| 31 | Saccharose | + |
| 32 | Trehalose | − |
| 33 | Inuline | − |
| 34 | Melezitose | − |
| 35 | D-Raffinose | + |
| 36 | Amidon | − |
| 37 | Glycogene | − |
| 38 | Xylitol | − |
| 39 | β Gentiobiose | − |
| 40 | D-Turanose | − |
| 41 | D-Lyxose | − |
| 42 | D-Tagatose | − |
| 43 | D-Fucose | − |
| 44 | L-Fucose | − |
| 45 | D-Arabitol | − |
| 46 | L-Arabitol | − |
| 47 | Gluconate | + |
| 48 | 2 ceto-gluconate | − |
| 49 | 5 ceto-gluconate | − |

Enzymatic properties (API ZYM KIT) of the selected strain Lactobacillus fermentum JS (patent deposit/KCCM-10499) are shown in Table 3 below.

TABLE 3

| | | |
|---|---|---|
| 1 | Control | − |
| 2 | Alkaline phosphatase | + |
| 3 | Esterase(C4) | + |
| 4 | Esterase Lipase(C8) | + |
| 5 | Lipase(C14) | − |
| 6 | Leucine arylamidase | + |
| 7 | Valine arylamidase | + |
| 8 | Crystine arylamidase | − |
| 9 | Trypsin | − |
| 10 | α-chymotrypsin | − |
| 11 | Acid phospatase | + |
| 12 | Naphtol-AS-Bl-phosphohydrolase | − |
| 13 | α-galactosidase | + |
| 14 | β-galactosidase | + |
| 15 | β-glucuronidase | − |
| 16 | α-glucosidase | − |
| 17 | β-glucosidase | − |
| 18 | N-acetyl-β-glucosaminidase | − |
| 19 | α-mannosidase | − |
| 20 | α-fucosidase | − |

### <Example 3> Confirmation of Synergistic Effects of Mixed Strains

To investigate the increase in ADH and ALDH activities of the Lactobacillus fermentum JS (patent deposited/KCCM-10499) strain when the Lactobacillus fermentum JS (patent deposited/KCCM-10499) strain selected in Examples 1 and 2 is mixed and applied with a strain without ADH and ALDH activities, the following mixed strain candidate compositions were used to implement experiments for measuring ADH and ALDH activities.

At this time, all the microorganisms used in the mixed strain were freeze-dried microorganisms, and the valid number thereof and a microorganism concentration were adjusted to 1.0×10⁹ cfu/g and used. Among separate strains except for the Lactobacillus fermentum JS (patent deposited/KCCM-10499) strain, only the strains having very little ability to increase ADH and ALDH activities were used.

A table for constitutional compositions of the mixed strains without ADH and ALDH is shown in Table 4 below.

TABLE 4

| | | Composition of mixed strain | | | |
|---|---|---|---|---|---|
| | | candidate (wt. %) | | | |
| Name of strain | Deposit no. | 1 | 2 | 3 | 4 |
| Lactobacillus | KCCM 10499 | 50 | 50 | 50 | 50 |
| fermentum JS | | | | | |
| Lactobacillus | KCCM 40018 | 10 | | | |
| plantarum | KCCM 12116 | | 10 | | 10 |
| | KCCM 40013 | | | 10 | |
| Lactobacillus casei | KCCM 12452 | 10 | | 10 | |
| | ATCC 4646 | | 10 | | 10 |
| Lactobacillus | KCCM 32826 | 8 | | | 8 |
| rhamnosus | ATCC 10863 | | 8 | | |
| | ATCC 393 | | | 8 | |
| Lactobacillus | KCCM 41270 | 6 | | | |
| acidophilus | ATCC 9224 | | 6 | 6 | |
| | KCCM 40265 | | | | 6 |
| Lactococcus lactis | KCCM 32406 | 6 | | 6 | |
| | ATCC 11454 | | | | 6 |
| | ATCC 11007 | | 6 | | |
| Bifidobacterium breve | ATCC 15700 | 5 | 5 | 5 | 5 |
| Bifidobacterium lactis | ATCC 27536 | 5 | | | 5 |
| | ATCC 700541 | | 5 | 5 | |

| | | | | |
|---|---|---|---|---|
| Sum | 100 | 100 | 100 | 100 |

ADH activity for each of the mixed strain candidates 1 to 4 was expressed by measuring the absorbance at 340 nm of NADH formed during decomposition of alcohol through a UV-Vis spectrophotometer. The strain was diluted 1/10 in distilled water to reach 1.0×10⁸ cfu/ml and used.

To a 15 ml Cornical tube, 100 μl of 99% alcohol, 500 μl of aqueous NAD solution, and 100 μl of the mixed strain candidate composition at a concentration of 1.0×10⁸ cfu/ml were added, followed by further adding 1.1 ml of 0.01M glycine-NaOH buffer (pH 8.8) thereto so as to make a total volume of 1.8 ml and then reacting the same in a constant-temperature water bath at 25° C. for 10 minutes. After the reaction was completed, 250 μl of ADH was added, followed by measuring a change in absorbance at 340 nm. At this time, for a control group, distilled water was added instead of the sample. A positive control group used herein was prepared by mixing 50% of Lactobacillus fermentum JS (patent deposit/KCCM-10499) at a concentration of 1.0×10⁹ cfu/g with 50% glucose and then diluting the same by 1/10 dilution. ADH activity was expressed as a ratio of the maximum absorbance at the end of the reaction to the maximum absorbance of the control group, and was calculated by the following equation.

ADH activity=(B/A)×100
A: maximum absorbance of the control group
B: maximum absorbance of the experimental group

Measurement of ALDH Activity

The ALDH activity was expressed by measuring the change in absorbance at 340 nm due to NADH generation, and the strain was diluted in distilled water by 1/10 to reach 1.0×10⁸ cfu/ml and used.

In a 15 ml Cornical tube, 2.1 ml of distilled water, 100 μl of 1M tris HCl, 100 μl of 3M KCl, 100 μl of the mixed strain candidate composition at a concentration of 1.0×10⁸ cfu/ml, 100 μl of NAD, 100 μl of 2-mercaptoethanol and 100 μl of acetaldehyde were mixed, followed by reacting the same at 25° C. for 10 minutes and then adding 100 μl of ALDH thereto, so as to measure a change in absorbance at 340 nm. At this time, for a control group, distilled water was added instead of the candidate strain. A positive control group used herein was prepared by mixing 50% of Lactobacillus fermentum JS (patent deposit/KCCM-10499) at a concentration of 1.0×10⁹ cfu/g with 50% glucose and then diluting the same by 1/10 dilution. ALDH activity was expressed as a ratio of the maximum absorbance at the end of the reaction to the maximum absorbance of the control group, and was calculated by the following equation.

ALDH activity=(B/A)×100
A: maximum absorbance of the control group
B: maximum absorbance of the experimental group

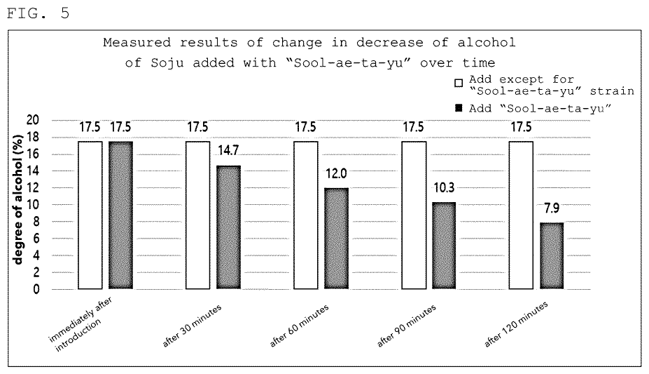

The measured results of ADH and ALDH activities of the mixed strains are shown in FIG. 4.

As a result of the experiment, it was confirmed that, when 7 strains having very little ADH activity and ALDH activity are mixed with the Lactobacillus fermentum JS (patent deposit/KCCM-10499) strain, the increase in ADH and ALDH activities is approximately 30% higher than that when using the Lactobacillus fermentum JS (patent deposited/KCCM-10499) alone, and there is very little influence on the used strain. The mixed strain has a constitutional composition in which 50 w/w % of the Lactobacillus fermentum JS (patent deposited/KCCM-10499) strain and, regardless of types of strains, 50 w/w % of seven (7) lactic acid bacteria including Lactobacillus plantarum, Lactobacillus casei, Lactobacillus rhamnosus, Lactobacillus acidophilus, Lactococcus lactis, Bifidobacterium breve and Bifidobacterium lactis are included. That is, it was found that the above composite lactic acid bacteria composition is effective in relieving hangover and bowel troubles.

In the case of the above 7 species of lactic acid bacteria, it was found that mixing 10 w/w % of Lactobacillus plantarum, 10 w/w % of Lactobacillus casei, 8 w/w % of Lactobacillus rhamnosus, 6 w/w % of Lactobacillus acidophilus, 6 w/w % of Lactococcus lactis, 5 w/w % of Bifidobacterium breve and 5 w/w % of Bifidobacterium lactis is effective in relieving hangovers and bowel troubles.

### <Example 4> Seed Culture of Selected Grain-Derived Lactic Acid Bacteria

A culture medium of the Lactobacillus fermentum JS (patent deposited/KCCM-10499) strain selected above is usually an MRS broth medium, and heat sterilization is performed at 121° C., which is the normal microbial medium sterilization condition, for 15 minutes or more. An inoculation amount of lactic acid bacteria was the concentration of 1×10³ to 1×10⁶ cfu/ml, and the above strain was cultured in the conditions of 160 rpm, pH 7.0 and to 45° C. for 12 to 18 hours. The culturing was conducted until the number of viable bacteria in the grain-derived lactic acid bacteria reaches the range of 1×10⁹ to 1×10¹⁰ cfu/ml after incubation.

### <Example 5> Preparation of Culture Solution

After placing 70 L of water in a 100 L culture tank, 5 w/v % of protease peptone No. 3, 2.5 w/v % of yeast extract, 10 w/v % of glucose, 4 w/v % of sodium acetate and 2 w/v % of ammonia citrate were added thereto, followed by sterilization at 121° C. for 15 minutes and then cooling to 40 to 45° C.

### <Example 6> Inoculation and Culture

The Lactobacillus fermentum JS (patent deposit/KCCM-10499) seeded in the culture medium prepared in Example 5 was inoculated to become a concentration of 1×10³ to 1×10⁶ cfu/ml and then incubated in the conditions of 160 rpm, pH 7.0, 40 to 45° C. for 12 to 18 hours, followed by culturing until the number of viable bacteria of the grain-derived lactic acid bacteria reaches the range of 1×10⁹ to 9×10⁹ cfu/ml after cultivation.

### <Example 7> Centrifugation, Lyophilization and Powderization of Grain-Derived Lactic Acid Bacteria

The Lactobacillus fermentum JS (patent deposit/KCCM-10499) strain, which was completely cultured at 1×10⁹ to 9×10⁹ cfu/ml, was centrifuged at 8000 rpm for 1 hour using a disc type centrifuge. Then, the centrifuged grain-derived lactic acid bacteria, that is, the Lactobacillus fermentum JS (patent deposited/KCCM-10499) strain concentrate as well as corn starch were mixed in a ratio of 50:50 (w/w %), and then freeze-dried and prepared for 3 days by any conventional method. Finally, using supplementary materials such as glucose, fructose, etc., the grain-derived lactic acid bacterium [Lactobacillus fermentum JS (patent deposited/KCCM-10499)] powder was prepared to reach a concentration of 1.0 to 1.2×10¹⁰ cfu/g.

### <Example 8> Mixing of Strains, Production of Composition “Sool-Ae-Ta-Yu” and Alcohol Decomposition Activity

By mixing the grain-derived lactic acid bacterium [Lactobacillus fermentum JS (patent deposited/KCCM-10499)] powder, which was completely prepared with the number of viable bacteria ranging from 1.0 to 1.2×10¹⁰ cfu/g, with other lactic acid bacteria at a concentration of 1.0 to 1.2×10¹⁰ cfu/g (purchased and used without cultivation) in the proportions shown in the table below, a strain mixture was prepared. At this time, the other strains mixed with the grain-derived lactic acid bacterium [Lactobacillus fermentum JS (patent deposit/KCCM-10499)] are used without distinguishing separate strains.

Table 5 shows a mixing ratio of the mixed strain powder.

TABLE 5

| Name of strain | Mixing ratio (w/w %) |
|---|---|
| Lactobacillus fermentum JS (KCCM 10499) | 50 |
| Lactobacillus plantarum | 10 |
| Lactobacillus casei | 10 |
| Lactobacillus rhamnosus | 8 |
| Lactobacillus acidophilus | 6 |
| Lactococcus lactis | 6 |
| Bifidobacterium breve | 5 |
| Bifidobacterium lactis | 5 |
| Sum | 100 |

The mixed strain was further blended with 20 w/w % of glucose, 14 w/w % of fructo-oligosaccharide, 14 w/w % of indigestible maltodextrin and 2 w/w % of palatinose with respect to 50 w/w % of the mixed strain, so as to produce a composition having a concentration of 5.0×10⁹ cfu/g as the final number of viable bacteria, which is named “Sool-ae-ta-yu (lactic acid bacteria to be ingested with alcoholic beverage)”.

Further, in order to investigate alcohol decomposition activity when the above composition “Sool-ae-ta-yu” is applied to Soju, Korean alcoholic beverage, which is actually sold in the market, the following experiment was conducted.

After purchasing a bottle (360 ml) of Soju commercially sold in the market (17.5% alcohol), it was left unopened at 37° C. for about 1 hour to allow the temperature of Soju to become in a state similar to that of a human body. Thereafter, 1.5 g of the composition “Sool-ae-ta-yu” was added to the above Soju, followed by shaking the same in a shaker incubator in the conditions of 37° C. and 30 rpm for 2 hours.

To investigate a change in alcohol concentration over time, 1 ml of sample was collected every 30 minutes from the Soju added with 1.5 g of “Sool-ae-ta-yu” in the conditions of 37° C. and 30 rpm, followed by measuring the alcohol concentration using a refractometer. At this time, a control group used herein was prepared by adding 1.5 g of a composition that has the same constitutional compositions as that of “Sool-ae-ta-yu” but contains dextrin added instead of the mixed strain, that is, except for “Sool-ae-ta-yu” strain. For the control group, a change in alcohol concentration was measured under the same conditions as described above.

The results are shown in FIG. 5.

According to FIG. 5, it could be seen that, when the product of the present invention is added to alcohol, the alcohol concentration is significantly reduced.

### <Example 9> Clinical Monitoring Results of Application of the Composition to Actual Soju for Each Age Group

In order to test effects of the composition on relief of hangovers, the composition with the brand name “Sool-ae-ta-yu” was supplied to panels drinking Soju at a bar. The panels have responded to the questionnaire results on relief of hangovers and bowel troubles on the next day after drinking, and the response results were statistically processed. Finally, the statistic results were classified only by age regardless of gender, and shown in <Table 6>, <Table 7>, <Table 8> and <Table 9>.

<Table 6> shows the response results of people in their 50's with respect to the hangover relief effects and bowel trouble relief.

As shown in the following <Table 6>, from the clinical monitoring results for the 50's people (50's panels: 190 people), the result of hangover relief after drinking demonstrates that the product of the present invention has very high hangover relief effects compared to the products of other companies. Further, it was found that effects of relieving intestinal problems, that is, bowel troubles such as diarrhea, abdominal pain and intestinal discomfort on the next day after drinking alcohol, were also very high. On the contrary, other companies' hangover relievers showed lower hangover relief effects than the hangover reliever of the present invention. In addition, it showed a low score with regard to relief of bowel troubles after drinking, demonstrating a result judged that bowel trouble relieving effects are almost unpredictable.

TABLE 6

| | Questionnaire | |
|---|---|---|
| | Result on hangover | Result on bowel |
| | relief effect after | trouble relief |
| Manufacturer/brand name | drinking | after drinking |
| The inventive product as | 4.9* ± 0.5*** | 4.8 ± 0.4^{a} |
| hangover reliever (brand name: | | |
| Sool-ae-ta-yu) | | |
| Hangover reliever of C Co. | 3.1 ± 0.3^{b} | 1.6 ± 0.4^{b} |
| Hangover reliever of S Co. | 3.3 ± 0.4^{b} | 1.8 ± 0.5^{b} |
| Hangover reliever of Y Co. | 2.0 ± 0.4^{c} | 1.7 ± 0.3^{b} |

*5 points: very high, 4 points: high, 3 points: moderate, 2 points: low, 1 point: very low
** As a value representing the statistical result, different vertical letters (lowercase letters ^{a}, ^{b}, and ^{c} in English) indicate significant results (p < 0.05).

<Table 7> shows the response results of people in their 40's to the hangover relief effect and bowel trouble relief.

The following <Table 7> is a result of targeting the age group of 40's (40's panels: 193 people), and showed similar results to the age group of 50's. That is, the product of the present invention showed a higher score than other manufacturers, thus demonstrating better effects in relieving hangover and bowel troubles on the next day after drinking.

TABLE 7

| | Questionnaire | |
|---|---|---|
| | Result on hangover | Result on bowel |
| | relief effect after | trouble relief |
| Manufacturer/brand name | drinking | after drinking |
| The inventive product as | 4.6 ± 0.6^{a} | 4.5 ± 0.5^{a} |
| hangover reliever (brand name: | | |
| Sool-ae-ta-yu) | | |
| Hangover reliever of C Co. | 3.0 ± 0.4^{b} | 2.1 ± 0.3^{b} |
| Hangover reliever of S Co. | 2.8 ± 0.4^{b} | 1.8 ± 0.4^{b} |
| Hangover reliever of Y Co. | 3.1 ± 0.3^{c} | 1.9 ± 0.2^{b} |

*5 points: very high, 4 points: high, 3 points: moderate, 2 points: low, 1 point: very low
** As a value representing the statistical result, different vertical letters (lowercase letters in English, ^{a}, ^{b}, and ^{c}) indicate significant results (p < 0.05).

<Table 8> shows the response results of people in their 30's to the hangover relief effect and bowel trouble relief.

The following <Table 8> is a result of targeting panels of 30's (30's panels: 197 people). Like as in <Table 6> and <Table 7>, the hangover reliever of the present invention showed a higher score, thus demonstrating better effects in relieving hangover and bowel troubles than those of other companies commercially sold in the market.

TABLE 8

| | Questionnaire | |
|---|---|---|
| | Result on hangover | Result on bowel |
| | relief effect after | trouble relief |
| Manufacturer/brand name | drinking | after drinking |
| The inventive product as | 4.4 ± 0.5^{a} | 4.5 ± 0.6^{a} |
| hangover reliever (brand name: | | |
| Sool-ae-ta-yu) | | |
| Hangover reliever of C Co. | 2.8 ± 0.3^{b} | 2.2 ± 0.1^{b} |
| Hangover reliever of S Co. | 2.6 ± 0.4^{b} | 2.4 ± 0.3^{b} |
| Hangover reliever of Y Co. | 2.9 ± 0.5^{c} | 2.2 ± 0.1^{b} |

*5 points: very high, 4 points: high, 3 points: moderate, 2 points: low, 1 point: very low
** As a value representing the statistical result, different vertical letters (lowercase letters in English, ^{a}, ^{b}, and ^{c}) indicate significant results (p < 0.05).

<Table 9> shows the response results of people in their 20's to the hangover relief effect and bowel trouble relief.

The following <Table 9> shows the effect on hangover relief and bowel troubles in the age group of 20's on the next day after drinking (20's panels: 195 people), and demonstrated similar results to other age groups. Further, it was found that the hangover relief effect of the product of the present invention is better than other companies' products. In addition, it was also confirmed that bowel trouble relief effects are also better as compared to other companies' products.

TABLE 9

| | Questionnaire | |
|---|---|---|
| | Result on hangover | Result on bowel |
| | relief effect after | trouble relief |
| Manufacturer/brand name | drinking | after drinking |
| The inventive product as | 4.3 ± 0.5^{a} | 4.1 ± 0.6^{a} |
| hangover reliever (brand name: | | |
| Sool-ae-ta-yu) | | |
| Hangover reliever of C Co. | 3.3 ± 0.4^{b} | 2.7 ± 0.3^{b} |
| Hangover reliever of S Co. | 3.5 ± 0.4^{b} | 2.9 ± 0.1^{b} |
| Hangover reliever of Y Co. | 3.5 ± 0.3^{c} | 2.5 ± 0.3^{b} |

*5 points: very high, 4 points: high, 3 points: moderate, 2 points: low, 1 point: very low
** As a value representing the statistical result, different vertical letters (lowercase letters in English, ^{a}, ^{b}, and ^{c}) indicate significant results (p < 0.05).

1. A composition for hangover relief, comprising: a grain-derived lactic acid bacterium [Lactobacillus fermentum JS (patent deposited/KCCM-10499)] strain, which has an ability of secreting alcohol dehydrogenase (“ADH”) and aldehyde dehydrogenase (“ALDH”) enzymes and/or increasing activities of these enzymes.

2. The composition according to claim 1, further comprising: one or more species of lactic acid bacteria selected from the group consisting of Lactobacillus plantarum, Lactobacillus casei, Lactobacillus rhamnosus, Lactobacillus acidophilus, Lactococcus lactis, Bifidobacterium breve and Bifidobacterium lactis, which exhibit synergistic effects when mixed with the grain-derived lactic acid bacterium [Lactobacillus fermentum JS (patent deposited/KCCM-10499)] strain although the ability of increasing activities of ADH and ALDH enzymes is a little low.

3. The composition according to claim 1, wherein the bacteria are living or dead bacteria.

4. The composition according to claim 1, wherein the composition is effective in relieving intestinal problems (or bowel troubles).

5. The composition according to claim 2, wherein the composition includes Lactobacillus plantarum, Lactobacillus casei, Lactobacillus rhamnosus, Lactobacillus acidophilus, Lactococcus lactis, Bifidobacterium breve and Bifidobacterium lactis.

6. The composition according to claim 2, wherein the composition includes: 40 to 60% by weight (“wt. %”) of the Lactobacillus Fermentum JS (KCCM-10499); and 40 to 60 wt. % of one or more species of lactic acid bacteria selected from the group consisting of Lactobacillus plantarum, Lactobacillus casei, Lactobacillus rhamnosus, Lactobacillus acidophilus, Lactococcus lactis, Bifidobacterium breve and Bifidobacterium lactis, to a total weight of the hangover relief composition.

7. The composition according to claim 6, wherein the composition includes: 40 to 60 wt. % of the Lactobacillus Fermentum JS; 8 to 12 wt. % of Lactobacillus plantarum; 8 to 12 wt. % of Lactobacillus casei; 6 to 10 wt. % of Lactobacillus rhamnosus; 4 to 8 wt. % of Lactobacillus acidophilus; 4 to 8 wt. % of Lactococcus lactis; 3 to 7 wt. % of Bifidobacterium breve; and 3 to 7 wt. % of Bifidobacterium lactis, to a total weight of the hangover relief composition.

8. A product for hangover relief, comprising the composition according to claim 1.

9. The hangover relief product according to claim 8, wherein the product includes: 40 to 60 wt. % of the hangover relief composition; and 40 to 60 wt. % of one or more selected from starch, glucose, fructo-oligosaccharide, indigestible maltodextrin and palatinose.

10. The hangover relief product according to claim 8, wherein the hangover relief product is added to an alcoholic beverage so as to decompose alcohol before drinking the alcoholic beverage.

11. The composition according to claim 2, wherein the bacteria are living or dead bacteria.

12. The composition according to claim 2, wherein the composition is effective in relieving intestinal problems (or bowel troubles).

13. A product for hangover relief, comprising the composition according to claim 2.
